(19) United States
(12) Patent Application Publication
(10) Pub. No.: US 2010/0312286 A1
(43) Pub. Date: Dec. 9, 2010

(54) Variable Angle Locked Bone Plate

(76) Inventor: Alberto A. Fernandez Dell'Oca, Montevideo (UY)

Correspondence Address: Fay Kaplun & Marcin, LLP, 150 Broadway, suite 702, New York, NY, 10038, US

(21) Appl. No.: 12/740,228

(22) PCT Filed: Oct. 30, 2008
(86) PCT No.: PCT/US08/81739

§ 371 Date: Apr. 28, 2010

Related U.S. Application Data
(60) Provisional application No. 61/000,907, filed on Oct. 30, 2007.
(60) Provisional application No. 61/084,281, filed on Jul. 29, 2008.

(51) Int. Cl. A61B 17/80

(52) U.S. Cl. 606/291

(57) ABSTRACT

A plate for fixation to a target portion of bone to be treated, comprises a first fixation element receiving opening extending therethrough from a proximal surface to a distal surface, the first fixation element receiving opening including a plurality of columns distributed about a circumference thereof and a plurality of radially expanded sections separating adjacent ones of the columns from one another in combination with a plurality of projections formed on the columns, the projections extending from surfaces of the columns, shapes of the surfaces of the columns on which the projections are formed being selected so that, when engaged by a head of a bone fixation element, the projections engage a thread of a head of the bone fixation element to lock the bone fixation element in the first fixation element receiving opening at any user selected angle within a permitted range of angulation.

### PRIORITY CLAIM

This application claims priority to (1) U.S. Provisional Application Ser. No. 61/000,907 entitled “Variable Angle Locked Bone Plate,” filed on Oct. 30, 2007 and (2) U.S. Provisional Application. Ser. No. 61/084,281, entitled “Variable Angle Locked Bone Plate,” filed on Jul. 29, 2008. The Specifications of the above-identified applications are incorporated herewith by reference.

### BACKGROUND

The present invention is directed to a locked bone fixation assembly, and in particular to an assembly that allows for a surgeon-selected angle of the bone screw relative to the fixation device.

Orthopedic fixation devices, both internal and external, are frequently coupled to bone using fasteners such as screws, threaded bolts or pins. For example, bone plates may be secured to bone with bone screws, inserted through plate holes. Securing the screws to the plate provides a fixed angle relationship between the plate and screw and reduces incidences of loosening. One method of securing the screw to the plate involves the use of so-called “expansion-head screws,” U.S. Pat. No. 4,484,570 discloses an expansion-head screw with a head that has a recess, the walls of which contain a number of slits. After an expansion-head screw has been inserted into a bone through a hole in the fixation device, a locking screw is inserted into the recess to expand the walls of the recess to lock the screw to the fixation device (such as a plate, internal fixator, nail, or rod). Another method of securing the screw to the plate involves the use of conical heads as shown in U.S. Pat. Nos. 5,053,036; 5,151,103; and 6,206,881, which disclose conical screw holes, adapted to receive screws having conical heads of a predetermined cone angle, such that the plate will not slide down the heads of the screws. A third method of securing the screw to the plate involves the use of so-called “locking screws,” A locking screw has threading on an outer surface of its head that matches with corresponding threading on the surface of a plate hole to lock the screw to the plate. Bone plates having threaded holes for accommodating locking screws are known, as shown in U.S. Pat. Nos. 5,709,686, and 6,730,091.

In addition to securing the screw to the fixation device, it is also often desirable to insert the screws at an angle relative to the fixation device selected by the surgeon. The prior art discloses a number of these so-called “polyaxial” systems, most of which utilize a bushing located in a hole in the fixation device to provide for locking at different degrees of angulation of the screw relative to the fixation device. For example, U.S. Pat. No. 5,954,722 discloses a polyaxial (selected variable axis) locking plate that includes a plate hole having a bushing rotatable within the hole. As a screw is inserted into bone through the bushing and plate hole, a threaded tapered head of the screw engages a threaded internal surface of the bushing to expand the bushing against the wall of the plate hole, thereby friction locking the screw at the desired angular orientation with respect to the plate. U.S. Pat. No. 6,575,975 discloses a polyaxial locking plate that includes a plate hole, having a a bushing rotatable within the hole, a fastening screw and a locking screw. The head of the fastening screw includes a radial wall that allows for outward expansion so that outwardly expanding the sidewall of the bushing so that the fastening screw is locked to the bushing and fixation device. A similar device is disclosed in U.S. Pat. No. 7,273,481.

Some others of the so-called “polyaxial” systems utilize a ring located in a hole in the fixation device. For example, U.S. Pat. No. 6,454,769 discloses a plate system and method of fixation comprising a bone plate, a bone screw and a ring, said ring being expandable against the bone plate to fix the bone screw at a selected angle relative to the bone plate.

These multi-component traditional plate assemblies can be cumbersome and tedious to manipulate during surgery to achieve the most desirable angle for directing the bone screw into the patient. U.S. Pat. No. 6,955,677 and U.S. Pat. Publ. Nos. 2005/0165400 and 2005/0277937 disclose additional polyaxial systems.

### SUMMARY OF THE INVENTION

The present invention is directed to a plate for fixation to a target portion of bone to be treated, comprising a first fixation element receiving opening extending therethrough from a proximal surface which, when the plate is coupled to the target portion of bone in a desired configuration, faces away from the bone to a distal surface which, when in the desired configuration, faces the bone, the first fixation element receiving opening including a plurality of columns distributed about a circumference thereof, each of the columns extending from the proximal to the distal surface and a plurality of radially expanded sections separating adjacent ones of the columns from one another in combination with a plurality of projections formed on the columns, the projections extending from surfaces of the columns along portions of a path extending helically about an inner surface of the first fixation element receiving opening, shapes of the surfaces of the columns on which the projections are formed being selected so that, when engaged by a head of a bone fixation element to be locked into the first fixation element receiving opening, the projections engage a thread of a head of the bone fixation element to lock the bone fixation element in the first fixation element receiving opening at any user selected angle within a permitted range of angulation.

### BRIEF DESCRIPTION OF THE DRAWINGS

FIG. 1 shows a perspective view of a bone fixation assembly according to the first embodiment of the present invention wherein a 4 holes bone plate and a threaded spherical screw prior to insertion in the bone plate are shown.

FIG. 2 is a perspective view of a spherical headed screw.

FIG. 3 is a front view of the bone fixation assembly with two separated screws, each of which locks in a different angle with respect to the plate, and wherein the bone plate was removed to best shown the locking position of the screw.

FIG. 4 is a perspective view of a plate hole according to the first embodiment of the present invention.

FIG. 5 is a perspective sectional view, at 1A-1A of FIG. 4, of the plate hole.

FIG. 6 is a front sectional view, at 1A-1A of FIG. 4, of the plate hole.

FIG. 7 is a perspective view of a bone fixation assembly according to the first embodiment of the present invention wherein the screw is perpendicularly locked to the bone plate, and wherein the anterior half of the plate has been shifted to the front to allow a better view of the locking system.

FIG. 8 is a perspective view of a bone fixation assembly according to the first embodiment of the present invention wherein the screw is locked at a tilt, and wherein the anterior half of the plate has been shifted to the front to allow a better view of the locking system.

FIG. 9 is a front view of a bone fixation assembly according to the first embodiment of the present invention wherein the screw is perpendicularly locked, and wherein the anterior half of the plate has been removed to allow a better view of the locking system.

FIG. 10 is a front view of a bone fixation assembly according to the first embodiment of the present invention wherein the screw is locked at a tilt, and wherein the anterior half of the plate has been removed to allow a better view of the locking system

FIG. 11 shows a perspective view of a bone fixation assembly according to the second embodiment of present invention wherein a 4 holes bone plate and a threaded spherical screw prior to insertion in the bone plate are shown.

FIG. 12 is a perspective view of a frusto-conical headed screw.

FIG. 13 is a front view of the bone fixation assembly with two separated screws, each of which locks in a different angle with respect to the plate, and wherein the bone plate was removed to best shown the locking position of the screw.

FIG. 14 is a perspective view of a plate hole according to the second embodiment of the present invention.

FIG. 15 is a perspective sectional view, at 1A-1A of FIG. 4, of the plate hole.

FIG. 16 is a front sectional view, at 1A-1A of FIG. 4, of the plate hole.

FIG. 17 is a perspective view of a bone fixation assembly according to the second embodiment of the present invention wherein the screw is perpendicularly locked to the bone plate, and wherein the anterior half of the plate has been shifted to the front to allow a better view of the locking system.

FIG. 18 is a perspective view of a bone fixation assembly according to the second embodiment of the present invention wherein the screw is locked at a tilt, and wherein the anterior half of the plate has been shifted to the front to allow a better view of the locking system.

FIG. 19 is a front view of a bone fixation assembly according to the second embodiment of the present invention wherein the screw is perpendicularly locked, and wherein the anterior half of the plate has been removed to allow a better view of the locking system.

FIG. 20 is a front view of a bone fixation assembly according to the second embodiment of the present invention wherein the screw is locked at a tilt, and wherein the anterior half of the plate has been removed to allow a better view of the locking system

### DETAILED DESCRIPTION

The present invention, which may be further understood with reference to the following description and the appended drawings, relates to devices for treating fractures and, in particular, to internal fixation devices for treating fractures. Exemplary embodiments of the present invention describe an improved bone fixation assembly that allows a surgeon to select an angle of a bone screw relative to a plate in a single action. The assembly uses only two components so that no rings, bushings or expansion head screws are needed. It will be understood by those of skill in the art that although the exemplary embodiments are described in regard to a screw/pin and a plate, the device may fix a fracture using any known fixation element.

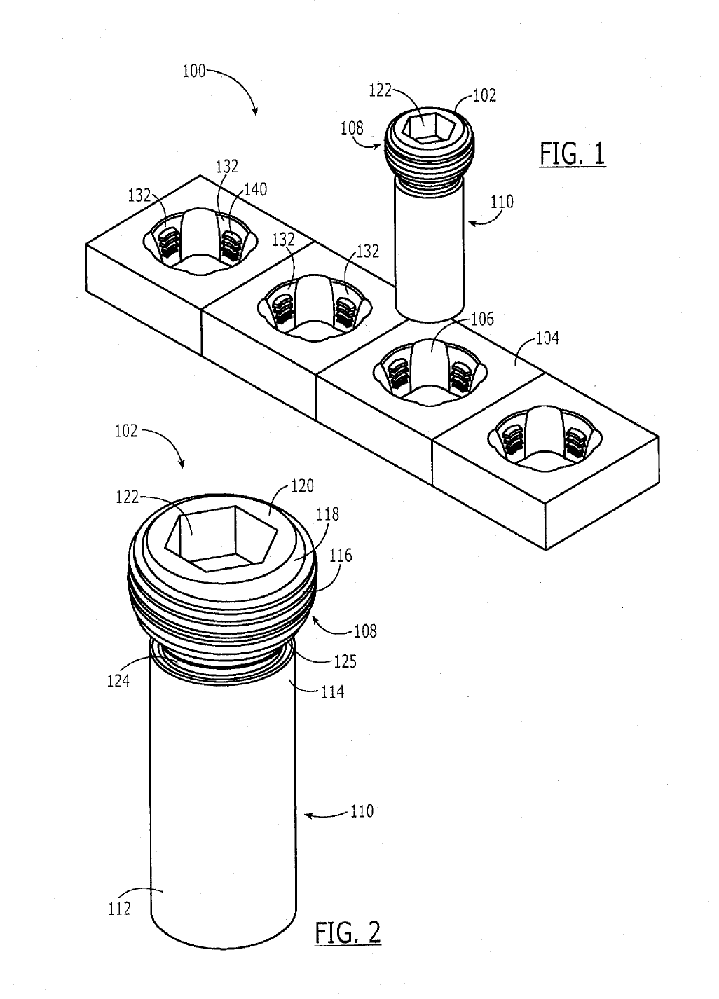

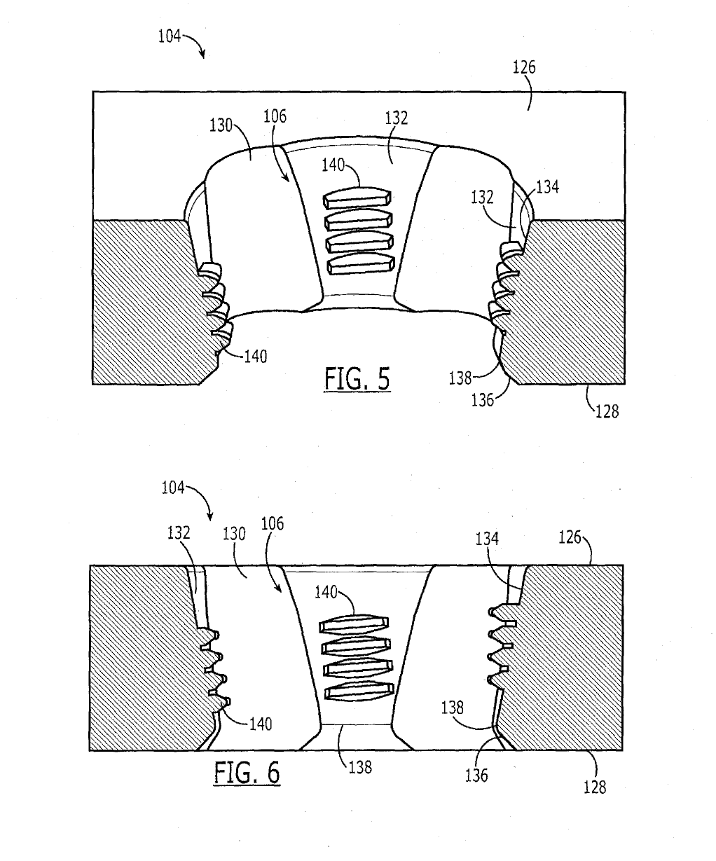

As shown in FIGS. 1-10, a device 100 according to a first exemplary embodiment of the invention comprises a screw or pin 102 and a plate 104. As shown in FIG. 1, the plate 104 includes at least one opening 106 receiving the screw or pin 102. As shown in FIG. 2, the screw or pin 102 further includes a head 108 and a shaft 110. The shaft 110 extends longitudinally from a distal end 112 to a proximal end 114. The head 108 is substantially spherical and includes a thread 116 extending around an outer surface 118 thereof at a pitch which, for example, may be substantially constant along the axis or along the radial surface. A proximal end 120 of the head 108 may include a driving element 122 for driving the screw or pin 102 through the opening 106 of the plate 104. The driving element 122 may, for example, be a hexagonal recess as shown, or any other recess or protrusion that may be engaged to facilitate the application of a torsional force to the screw or pin 102 to rotate the screw or pin 102 about a central axis thereof to drive the screw or pin 102 through the opening 106. The screw or pin 102 may further include a neck portion 124 connecting a distal end 125 of the head 108 to the proximal end 114 of the shaft 110. A diameter of the neck portion 124 is smaller than a diameter of both the head 108 and the shaft 110 to enhance the range of angulation of the screw or pin 102 relative to the plate 104 as will be described in more detail below. It will be understood by those of skill in the art that a thread may optionally be provided along a length of the shaft 110 of the screw or pin 102 as with conventional bone screws. It will also be understood by those of skill in the art that the thread 116 may extend around both the outer surface 118 of the head 108 and along a length of the shaft 110 from the proximal end 114 to the distal end 112 at a pitch selected to facilitate insertion of the screw or pin 102 into the bone as the head 108 is screwed into the plate 104.

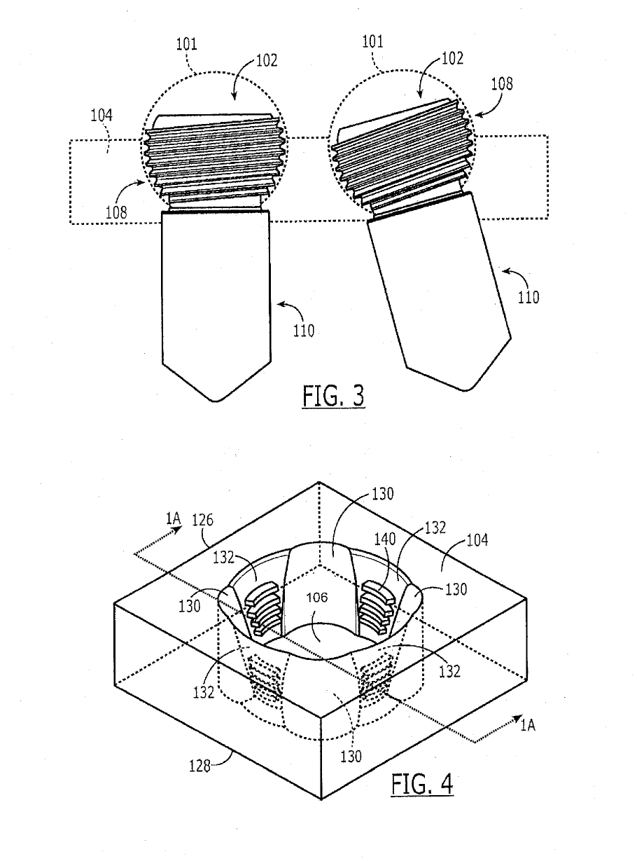

As shown in FIG. 3, the opening 106 of the plate 104 is adapted and configured to receive the head 108 of the screw or pin 102 at any angle relative to a central axis of the opening 106 within a permitted range of angulation. The screw or pin 102 may be inserted coaxially with the opening 106 or at any angle relative to the central axis of the opening 106 ranging from 0° to 45°. In an alternative embodiment, the range of angulation is from 0° to approximately 15° relative to the central axis of the opening 106. Since the head 108 is substantially spherical, varying the angle of insertion of the screw or pin 102 rotates the head 108 a circular pattern 101. As shown in FIGS. 4-6, the opening extends through the plate 104 from a proximal surface 126 thereof which faces away from a bone to a distal, bone facing surface 128. The opening 106 includes a plurality of scalloped portions 130 (in this embodiment 4 scalloped portions 130) disposed about a circumference of the opening 106 and separating a plurality of columns 132 from one another. Each of the columns 132 has a complex shape including a first portion 134 tapering radially inward toward the central axis of the opening 106 from the proximal surface 126 to a distal end 138 at which the first portion 134 is coupled to a second portion 136 tapering radially outward from the distal end of the first portion 134 to the distal surface 128 of the plate 102. The first portions 134 of the columns 132 are arranged along a first substantially conic shape centered on the central axis of the opening 106 and the second portions 136 of the columns 132 are arranged along a second substantially conic shape centered on the central axis of the opening 106. The scalloped portions 130 between these columns 132 are, for example, substantially cylindrical additions to the opening 106 extending radially outward beyond the first and second conic shapes extending the range of angulation of a screw or pin 102 inserted through the opening 106 as will be described in more detail below.

In an exemplary embodiment, the opening 106 may include four columns 132, spaced about the circumference of the opening 106 substantially equidistant from one another with widths of the scalloped portions 130 being substantially equal to one another. It will be understood by those of skill in the art however, that the opening 106 may include any number of columns 132 arranged in any number of patterns. Furthermore, those skilled in the art will understand that the columns 132 do not have to be equidistant from one another (i.e., they may be spaced about the circumference of the opening 106 by varying distances) and they may have different widths as well.

The first portion 134 preferably extends radially inward from the proximal surface 126 at an angle corresponding to the maximum angulation of the screw relative to the central axis of the opening. In this embodiment, this angle may be between 0° to 45° or, more preferably, between 0° and 15° depending on the desired angulation. The first portion 134 further includes a plurality of columns of threads 140 extending from a surface of the first portion 134 into the opening 106. Each column 132 may include at least two individual threads 140. However, it will be understood by those of skill in the art that the columns 132 may include any number of threads 140. The threads 140 are adapted and configured to engage the threading 116 of the head 108 and extend, for example, along paths which, if continued across the gaps formed by the scalloped portions 130, would form a helical threading with a substantially constant pitch corresponding to the threading 116 of the head 108 of the screw or pin 102. Alternatively, the threads 140 on each of the columns 132 may be positioned along the first portion 134 arranged substantially symmetrically with respect to the threads 140 of the other columns 132. Furthermore, the threads 140 are rounded to facilitate engagement with the threading 116 of the head 108 of the screw or pin 102 as would be understood by those skilled in the art.

The second portion 136 extends radially outward from the distal end 138 of the first portion 134 toward the distal surface 128 of the plate 104 at an angle ranging from 0° to 45°, but preferably approximately 15° relative to the central axis of the opening 106 such that the substantially conic portion formed by the second portions 136 of the plurality of columns 132 is adapted and configured to accommodate the proximal end 114 of the shaft 110 at varying angles. A length of the first portion 134 may be substantially longer than a length of the second portion 136, permitting the head 108 of the screw 102 to be fixed within the opening 106 of the plate 102.

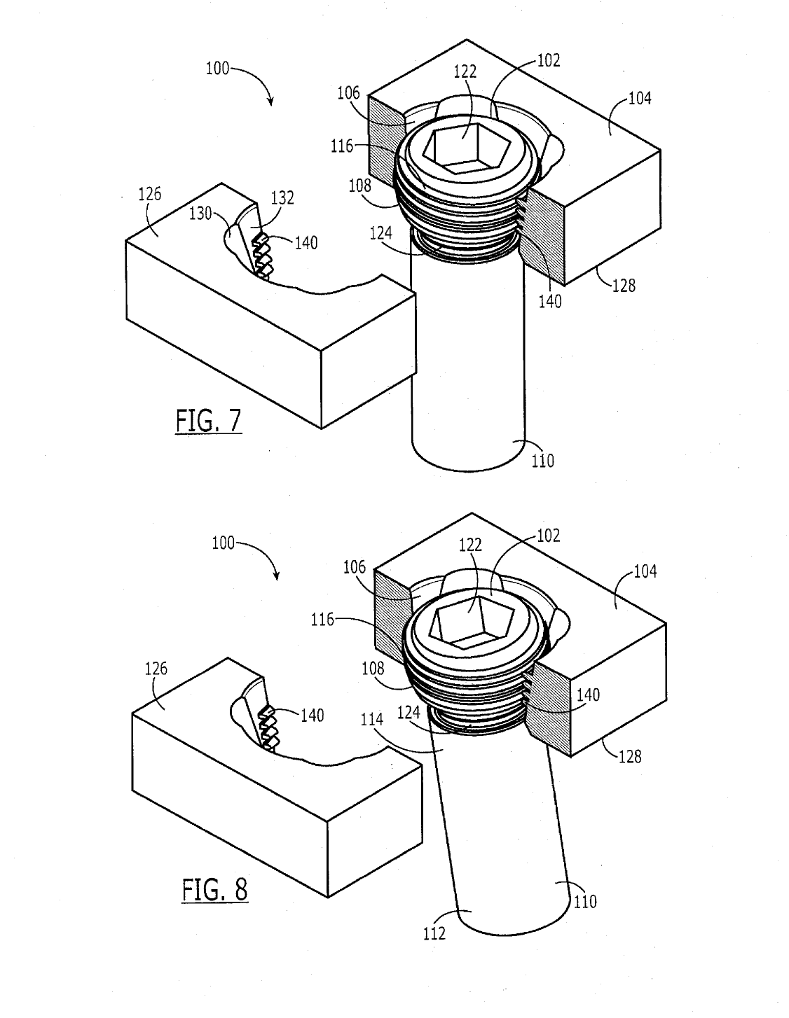

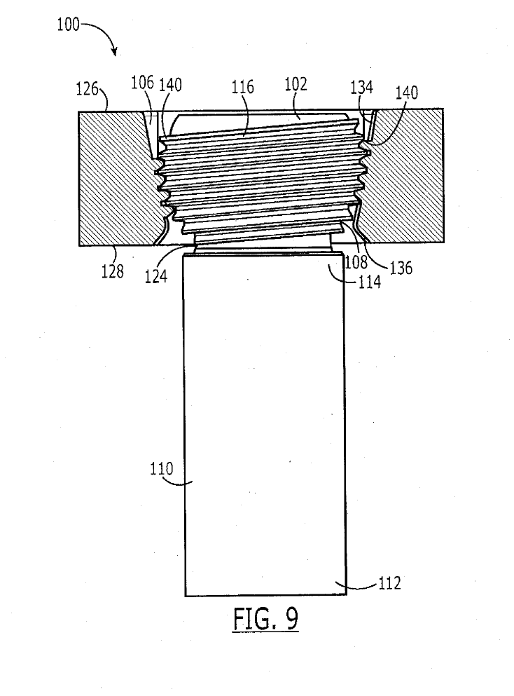

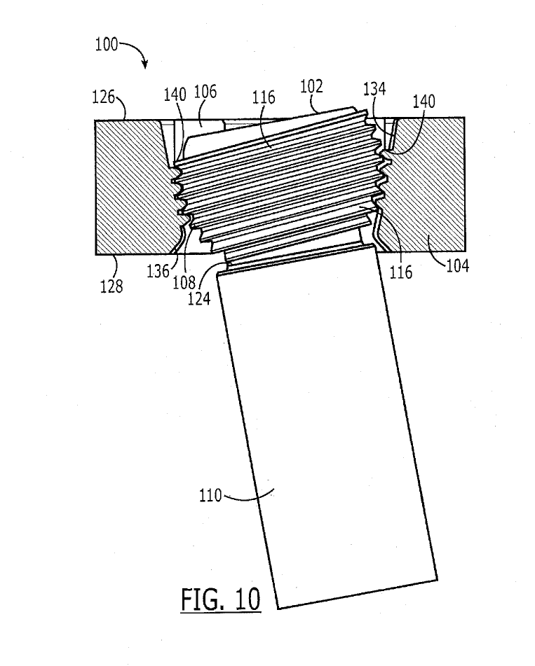

As shown in FIGS. 7-10, the spherical shape of the screw head 108 permits the threading 116 of the screw head 108 to engage the threads 140, whether inserted co-axially with the central axis of the opening 106, as shown in FIGS. 7 and 9, or offset from the central axis, as shown in FIGS. 8 and 10. Depending on the number of threads 140 and the angle of the screw 102, it will be understood by those of skill in the art that the threading 116 of the head 108 may not engage all of the threads 140. The neck portion 124, which may be of a smaller diameter than either of the head 108 and the shaft 110, may also accommodate the distal end 138 of the first portion 134 of the column 132 toward which the shaft 110 is angled. As would be understood by those skilled in the art, this forms a recess into which the shaft 110 is received permitting increased angulation of the screw 102 relative to the central axis of the opening 106.

In use, a plate 104 as described above is located in a desired position adjacent to a portion of bone to be stabilized. As indicated above, depending upon the bone being treated, it may be desirable to insert a screw or pin 102 through the opening 106 co-axially with the opening 106 or at an angle offset from the central axis of the opening 106. For example, the desired angle of insertion for each of a plurality of screws and/or pins 102 may be determined prior to insertion of the screw or pin 102 to achieve a desired effect on the target portion of bone. Each screw or pin 102 is then inserted into the opening 106 at its desired angle so that the threading 116 of the head 108 engages the threads 140 fixing the screw or pin 102 at this angle relative to the central axis of the opening 106 to fix the plate 104 to the target portion achieving any desired angle locking, etc.

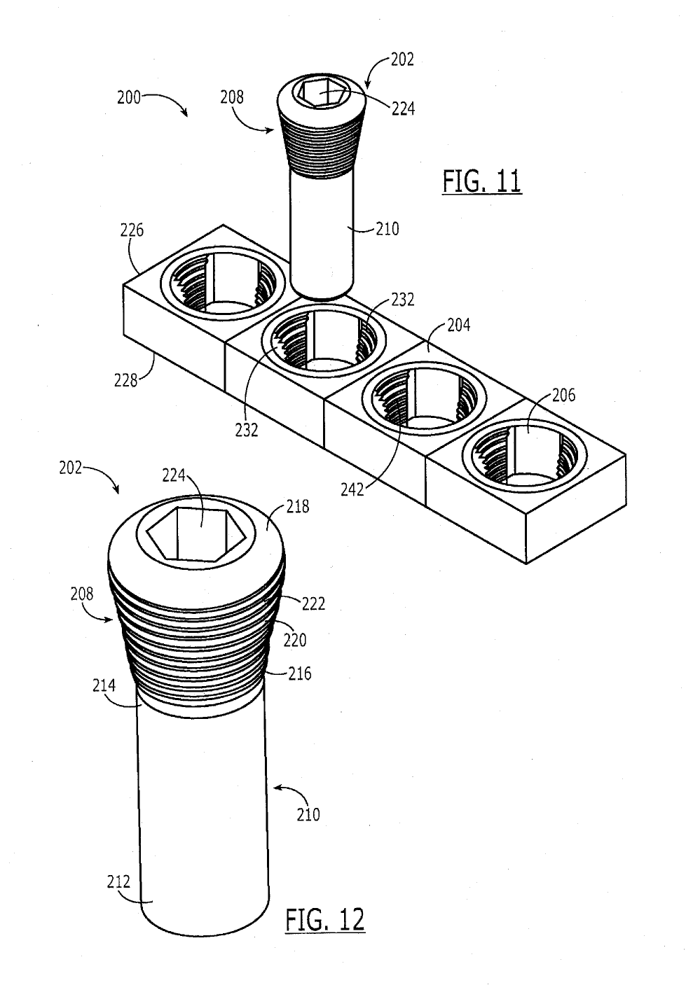

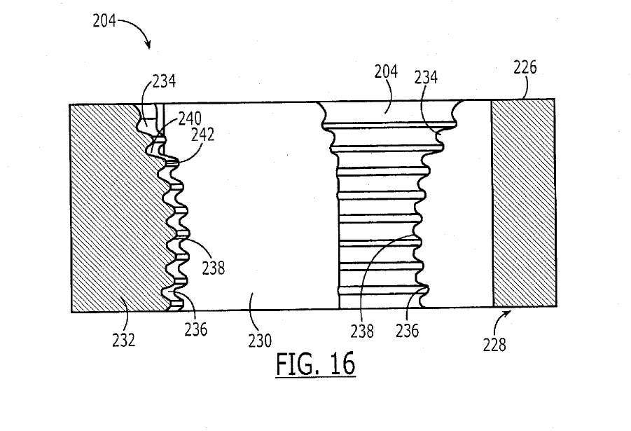

As shown in FIGS. 11-20, a device 200 according to a second exemplary embodiment of the present invention includes a plate 204 adapted for use with a screw or pin 202 which, in place of the spherical head of the screw or pin 102 has a substantially conical head 208. The device 200 may be substantially similar to the device 100, as described above except for the aspects of the geometry of the openings 206 in the plate 204 and the geometry of the head 208 of the screw or pin 202 as will be described below. As shown in FIG. 11, the plate 204 includes at least one opening 206 (in this case four openings 206) adapted and configured to receive the screw or pin 202 and to lock the screw or pin 202 at a desired angle relative to a central axis of the opening 206 into which it is inserted. As shown in FIG. 12, the screw 202 includes a head 208 and a shaft 210. The shaft 210 extends from a distal end 212 to a proximal end 214 with the head 208 formed at the proximal end 214 of the shaft 210. Unlike the screw or pin 102, however, the head 208 of the screw or pin 202 is substantially conical with a proximal end 218 of the head 208 having a larger diameter than a distal end 216 of the head 208. The conical head 208 tapers from the proximal end 218 to the distal end 216, relative to a central axis of the screw or pin, at an angle ranging between 5 degrees and 15 degrees, but preferably at an angle of approximately 10 degrees.

The head 208 includes a thread 220 formed along an outer surface 222 thereof. The thread 220 may extend from the proximal end 218 to the distal end 216 of the head 208 at a substantially constant pitch to lockingly engage an openings 206. The head 208 may also include a driving element 224 at the proximal end 218, which may be engaged by a driving tool to drive the screw or pin 102 through the opening 206 by rotating the screw or pin 102 about a longitudinal axis of the screw or pin 102. It will be understood by those of skill in the art that although the driving element 224 is shown as a hexagonal recess, the driving element 224 may take any of a variety of shapes and forms. For example, the driving element 224 may be any recess or protrusion so long as the driving element 224 may be engaged by a driving tool to drive the screw or pin 102 into the opening 106.

The distal end 216 of the head 208 may be connected to the proximal end 214 of the shaft 210, but need not include a neck portion with a smaller diameter, as described above in regard to screw or pin 102. The screw or pin 202, does not require an undercut to accommodate any portion of the opening 206 when the screw or pin 202 is angled relative to the central axis of the opening 204. The shaft 210 may also include a threading along any portion of a length of the shaft 210 if desired. As would be understood by those skilled in the art, the threading of the shaft 210 may also be formed at a substantially constant pitch which may be substantially equal to that of the thread 220 so that, as the screw 202 is rotatably threaded into the opening 206, the thread of the shaft 210 advances at the same rate into the bone.

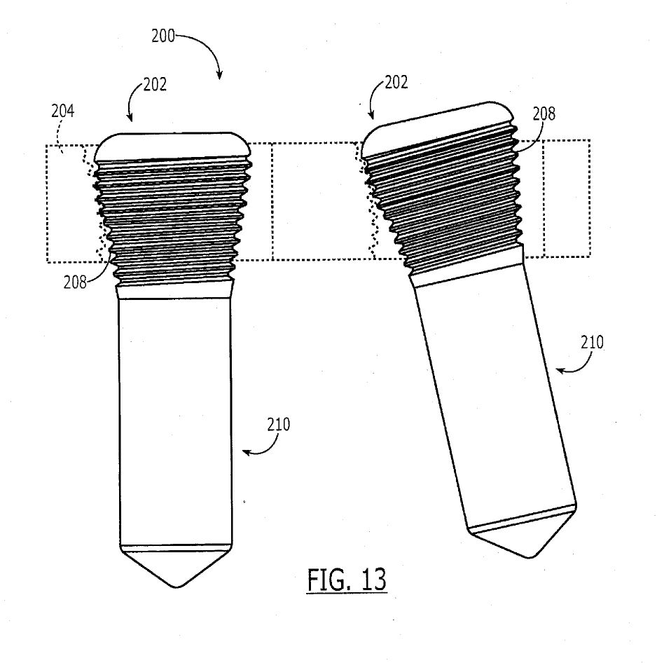

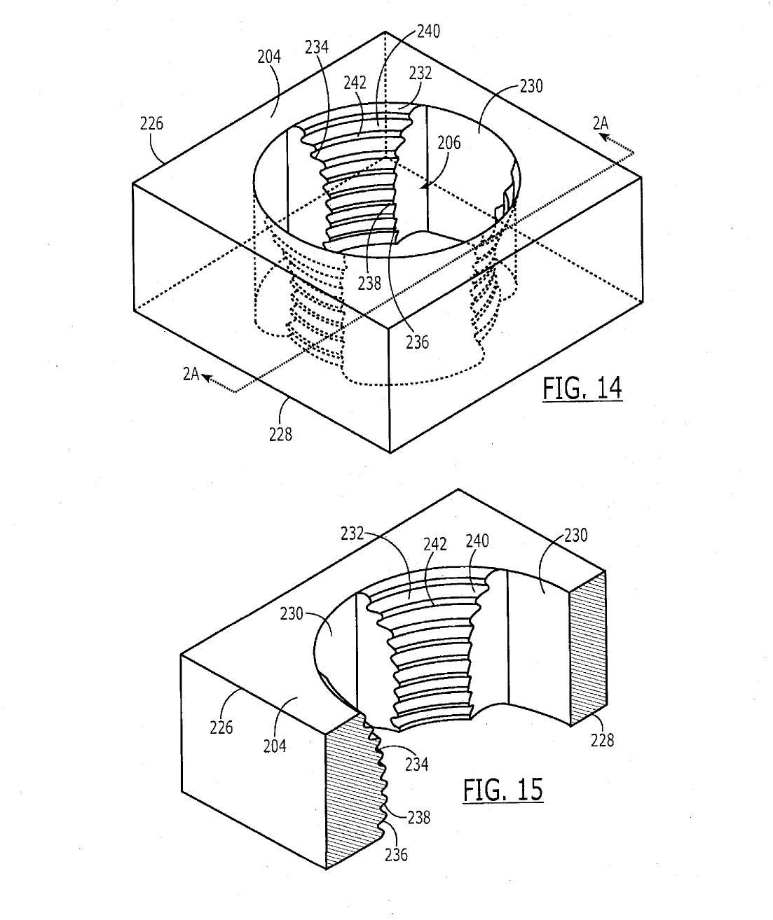

As shown in FIG. 13, the screw or pin 202 may be received within the opening 206 either co-axially with a central axis of the opening 206 or offset at an angle relative to the central axis. The thread 220 of the head 208 then engages a portion of the opening 206 as will be described below to fix the screw or pin 202 to the plate 204 at the desired angle. The screw or pin 202 may be inserted into the opening 206 at an angle ranging from 0° to 20°, but preferably up to 15°. As shown in FIGS. 14-16, the opening 206 extends from a proximal surface 226 of the plate 204, which faces away from a bone on which the plate 204 is mounted, to a distal surface 228 which faces the bone. Similarly to the plate 104, the opening 206 of the plate 204 includes a plurality of scalloped portions 230 disposed about a circumference thereof separating a plurality of columns 232 from one another. Each of the columns forms part of a surface which, if not separated into sections by the scalloped portions 230, would be circular in cross-section in planes perpendicular to the central axis of the opening 206. Rather than including angled first and second portions, each of the columns 232 has a complex shape extending over a curved surface 240 including a proximal portion 234 adjacent to the proximal surface 226 and a distal portion 236 adjacent to the distal surface 228. The proximal portion 234 curves radially inward (toward a central axis of the opening 206) as it extends from the proximal surface 226 toward a distal end 238 thereof while the distal portion 236 curves radially outward (away from the central axis of the opening 206) from the distal end 238 of the proximal portion 234 to the distal surface 228. The curved surface 240 forms a substantially continuous convex shape extending radially into the opening 206 toward a portion of the opening 206 between the distal and proximal surfaces 228, 226, respectively, and moving radially outward toward each of the distal and proximal surfaces 228, 226, respectively. The scalloped portions 230 between the columns 232 may be formed, for example, as substantially cylindrical additions extending radially outward

In a preferred embodiment, the opening 206 may include three columns 232 and three scalloped portions 230 with the columns 232 evenly spaced about the circumference of the opening 206 and the scalloped portions 230 placed between adjacent ones of these columns 232. It will be understood by those of skill in the art, however, that any number of columns 232 and scalloped portions 230 may be employed within the opening 206 so long as the resulting structure permits the desired angulation of the screws or pins 202 therein.

The curvature of the column 232 may be selected based on a variety of factors including, but not limited to, a size of the opening 206, a maximum desired tilt angle of the screw or pin 202 relative to a central axis of the opening 206 and the taper angle of the head 208 of the screw or pin 202. It will be understood by those of skill in the art that an angle of a tangent to the curvature of the proximal portion 234 of the column may be larger than an angle of a tangent of the curvature at the distal portion 236 of the column 232 to accommodate the taper of the head 208. The angle of the tangent to the proximal portion 234 of the column 232 may be determined by adding the maximum desired tilt of the screw or pin 202 relative to the central axis of the opening 206 to the taper angle of the head 208. For example, if the taper angle of the head 208 is 10° and the maximum desired tilt of the screw or pin 102 is 15°, the proximal portion 234 should be formed so that a tangent thereto is at an angle of 25° relative to the central axis. The angle of the tangent to the distal portion 236 may be determined by subtracting the taper angle of the head 208 from the maximum desired tilt angle of the screw or pin 202. Thus, using the same preferred tilt and taper angles described above, the distal portion 236 should be formed to provide a tangent to the surface angled 5° relative to the central axis of the opening 206.

Similar to the opening 106 described above, each of the columns 232 includes a column of threads 242 extending thereacross and separated from one another along a length of the column 232 from the proximal surface 226 to the distal surface 228. The threads 242 extend into the opening 206 in the same manner as a thread would on a known threaded opening. In one embodiment, each thread 242 extends into the opening 206 substantially perpendicularly to a tangent to the surface of the column 232 in a manner similar to the projection of gear teeth from a curved surface while, in an alternate embodiment, each of the threads 242 extends into the opening 106 in substantially parallel planes (e.g., in planes substantially perpendicular to the central axis of the opening 206). Similarly to the threads 140 of the opening 106, the threads 242 extend across each of the columns 232 along paths which, if not separated by the scalloped portions 230, would form a helical path with a pitch selected to facilitate insertion of the screw or pin 102 into the bone as the head 208 is screwed into the opening 206. It will be understood by those of skill in the art, however, that the threads 242 may be positioned in any of a variety of patterns, so long as the threads 242 engage the thread 220 of the head 208, locking the head 208 of the screw or pin 202 in a desired orientation within the opening 206.

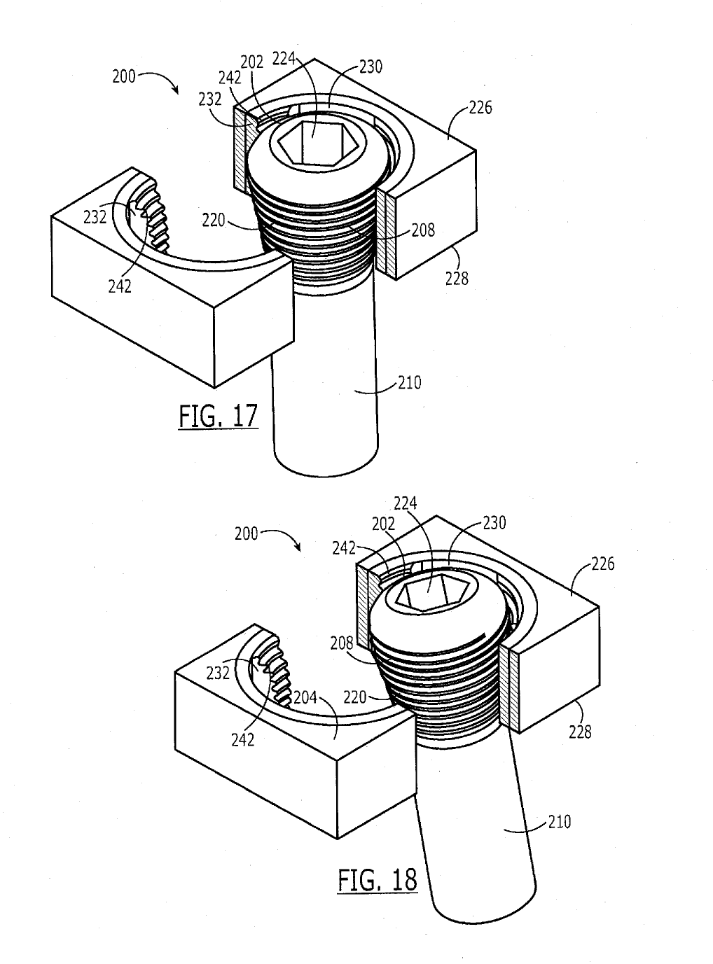

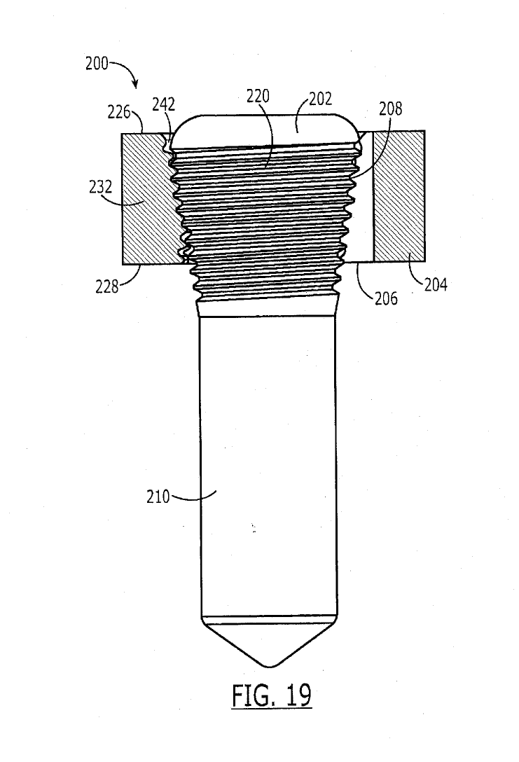

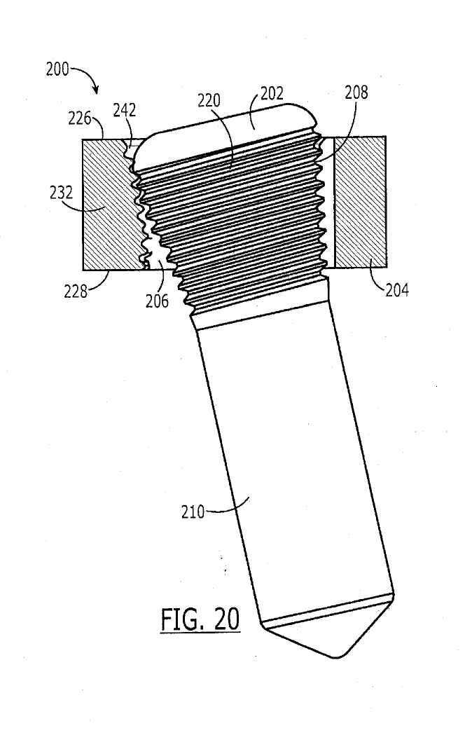

As shown in FIGS. 17-20, the conical shape of the head 208 allows the thread 220 of the head 208 to engage a portion of the threads 242 across the entire range of desired angulation of the screw or pin 202 relative to the central axis of the opening 206, whether co-axial with the central axis of the opening 206, as shown in FIGS. 17 and 19, or offset at an angle relative to the central axis, as shown in FIGS. 18 and 20. The device 200 may be used in substantially the same manner as described above in regard to the device 100. Additionally, as will be understood by those skilled in the art, the screw or pin 202 may be used with the plate 104 of the device 100 such that the threading 220 of the conical head 208 engages with the threads 140 of the plate 104 when inserted either co-axially with the opening 106 or at an angle relative to the central axis of the opening 106.

It will be apparent to those skilled in the art that various modifications and variations can be made in the structure and the methodology of the present invention, without departing from the spirit or scope of the invention. Thus, it is intended that the present invention cover the modifications and variations of this invention provided that they come within the scope of the appended claims and their equivalents.

What is claimed is:

1. A plate for fixation to a target portion of bone to be treated, comprising:
a first fixation element receiving opening extending therethrough from a proximal surface which, when the plate is coupled to the target portion of bone in a desired configuration, faces away from the bone to a distal surface which, when in the desired configuration, faces the bone, the first fixation element receiving opening including:
a plurality of columns distributed about a circumference thereof, each of the columns extending from the proximal to the distal surface;
a plurality of radially expanded sections separating adjacent ones of the columns from one another; and
a plurality of projections formed on the columns, the projections extending from surfaces of the columns along portions of a path extending helically about an inner surface of the first fixation element receiving opening, shapes of the surfaces of the columns on which the projections are formed being selected so that, when engaged by a head of a bone fixation element to be locked into the first fixation element receiving opening, the projections engage a thread of a head of the bone fixation element to lock the bone fixation element in the first fixation element receiving opening at any user selected angle within a permitted range of angulation.

2. The device of claim 1, wherein the first fixation element receiving opening is sized and shaped to lock therein a bone fixation element at a desired angle with respect to a central axis of the first fixation element receiving opening, the projections being separated along the central axis of the first fixation element receiving opening by a distance corresponding to a pitch of a helical thread on a head of the bone fixation element so that a portion of the projections threadingly engage the helical thread of the head of the bone fixation element.

3. The device of claim 1, wherein each of the columns includes a first portion tapering radially inward from the proximal surface to a deflection point within the first fixation element receiving opening, each of the columns including a second portion tapering radially outward from the deflection point to the distal surface so that a cross-sectional area of the first fixation element receiving opening is smaller at the deflection point than at the proximal and distal surfaces.

4. The device of claim 1, wherein a first one of the radially expanded sections is formed as substantially cylindrical lumen extending substantially parallel to the central axis of the first fixation element receiving opening.

5. The device of claim 4, wherein cross-sections of portions of the first fixation element receiving opening formed by the columns in planes perpendicular to the central axis of the first fixation element receiving opening are substantially circular and wherein the first radially expanded section extends radially beyond a diameter of the portions of the first fixation element receiving opening at the proximal and distal surfaces.

6. The device of claim 1, wherein the shape of the surfaces of the columns on which the projections are formed is formed to threadedly engage a thread formed on a substantially conical head of a bone fixation element to be locked into the first fixation element receiving opening at a user selected angle within the permitted range of angulation.

7. The device of claim 1, wherein the shape of the surfaces of the columns on which the projections are formed curves in a plane parallel to the central axis of the first fixation element receiving opening so that angles of tangents to the curves adjacent to the proximal surface with respect to the central axis of the central first fixation element receiving opening are equal to a maximum desired angulation of a bone fixation element to be locked in the first fixation element receiving opening plus a taper angle of a conical head of the bone fixation element.

8. The device of claim 7, wherein the tangents to the curves adjacent to the proximal surface ranges from 25° to 35°.

9. The device of claim 8, wherein the range of angulation of a bone fixation element to be locked in the first fixation element receiving opening is from 0° to 45°.

10. The device of claim 7, wherein each of the projections extends outward from the surface of the corresponding column substantially perpendicular thereto.

11. The device of claim 7, wherein each of the projections extends outward from the surface of the corresponding column substantially perpendicular to the central axis of the first fixation element receiving opening.

12. The device of claim 7, wherein an angle of a tangent of a curvature of a proximal portion of each column is a sum of a maximum angular offset of a bone fixation element to be locked into the first fixation element receiving opening and a taper angle of a conical head of the bone fixation element.

13. The device of claim 12, wherein an angle of a tangent of a curvature of a distal portion of each column is a difference between the maximum angular offset of a bone fixation element to be locked into the first fixation element receiving opening and the taper angle of the conical head of the bone fixation element.

14. The device of claim 1, wherein the columns are distributed about the circumference of the first fixation element receiving opening equidistant from one another.

15. A method, comprising:
positioning a plate over a target portion of bone to be treated, the plate; and
inserting a fixation element into the bone via an opening extending through the plate at a user selected angle relative to a central axis of the opening, the opening including a plurality of columns distributed about a circumference of the opening and a plurality of radially expanded sections separating adjacent ones of the columns from one another; and
threadingly engaging a thread of a head of the fixation element with a plurality of projections extending from surfaces of the columns to lock the fixation element in the opening at the user selected angle, the projections extending along portions of a path extending helically about an inner surface of the opening.
